(19) United States
(12) Patent Application Publication
(10) Pub. No.: US 2009/0162442 A1
(43) Pub. Date: Jun. 25, 2009

(54) MULTI-PHASIC, NANO-STRUCTURED COMPOSITIONS CONTAINING A COMBINATION OF A FIBRATE AND A STATIN

(76) Inventors: Dinesh B. Shenoy, Karnataka State (IN); Robert Lee, Boyertown, PA (US)

Correspondence Address: COOLEY GODWARD KRONISH LLP;ATTN: Patent Group, Suite 1100, 777 - 6th Street, NW, WASHINGTON, DC, 20001, US

(21) Appl. No.: 12/206,264

(22) Filed: Sep. 8, 2008

Related U.S. Application Data
(60) Provisional application No. 60/970,684, filed on Sep. 7, 2007.

(51) Int. Cl. A61K 9/16; A61K 31/40; A61P 3/06; A61K 31/435; A61K 31/404; A61K 31/35; A61K 31/47; A61K 31/505

(52) U.S. Cl. 424/489; 514/427; 514/277; 514/415; 514/460; 514/311; 514/275

(57) ABSTRACT

The present invention discloses a pharmaceutical formulation containing a multi-phasic pharmaceutical composition in an oral dosage form. The multi-phasic pharmaceutical composition contains: (a) a fibrate, or a pharmaceutically acceptable salt, ester, hydrate, or prodrug thereof; (b) a statin, or a pharmaceutically acceptable salt, ester, hydrate, or prodrug thereof; (c) a solvent; (d) a non-miscible liquid; (e) a stabilizer; and (f) water. The fibrate or the statin or both is in a particulate state and/or a solubilized state. Such pharmaceutical formulations are capable of reducing the fed/fast variability and improving oral bioavailability to which a number of active pharmaceutical ingredients are susceptible. The pharmaceutical formulations of the invention, therefore are bioequivalent in fed and fasted states and have improved oral bioavailability.

### CROSS-REFERENCE TO RELATED APPLICATIONS

This application claims priority to U.S. Provisional Patent Application Ser. No. 60/970,684, entitled “Multi-phasic, Nano-structured Compositions Containing a Combination of a Fibrate and a Statin”, filed Sep. 7, 2007, the content of which is herein incorporated by reference in their entirety for all purposes. This application is also related to U.S. Provisional Patent Application Ser. No. 60/881,470, entitled “Multi-phasic Pharmaceutical Formulations of Poorly Water-soluble Drugs for Reduced Fed/Fasted Variability and Improved Oral Bioavailability”, filed Jan. 22, 2007; and U.S. Provisional Patent Application Ser. No. 60/857,511, entitled “Method of Preparing Solid Dosage Forms of Multi-phasic Pharmaceutical Compositions”, filed Nov. 8, 2006, the content of which is herein incorporated by reference in their entirety for all purposes.

### FIELD OF THE INVENTION

The present invention generally relates to multi-phasic compositions containing a fibrate and a statin and methods of making the same. More particularly, these multi-phasic compositions may be used to reduce fed-fasted absorption variability and improve oral bioavailability of the active pharmaceutical ingredients.

### BACKGROUND OF THE INVENTION

Hyperlipidemia, also known as hyperlipoproteinemia or dyslipidemia refers to the presence of elevated or abnormal levels of lipids and/or lipoproteins in the blood. Combined hyperlipidemia is a commonly occurring form of hypercholesterolemia and hypertriglyceridemia characterized by increased low-density lipoprotein (LDL) and triglyceride concentrations, often accompanied by decreased low-density lipoprotein (HDL). The elevated triglyceride levels (>5 mmol/l) are generally due to an increase in very low density lipoprotein (VLDL), a class of lipoprotein that is prone to cause atherosclerosis.

Lipid and lipoprotein abnormalities are very common in the general population and pose a serious public health concern. Hyperlipidemia is regarded as a high risk factor for cardiovascular disease due to the influence of cholesterol, one of the most clinically relevant lipid substances, on atherosclerosis. Hyperlipidemia most commonly becomes seriously symptomatic when interfering with the coronary circulation supplying the heart or cerebral circulation supplying the brain, and is considered the most important underlying cause of strokes, heart attacks, various heart diseases including congestive heart failure and most cardiovascular diseases in general. According to United States data for the year 2004, for about 65% of men and 47% of women, the first symptom of atherosclerotic cardiovascular disease is heart attack or sudden cardiac death (death within one hour of onset of the symptom).

Various medicines, particularly hypolipidemic agents, such as fibrates and statins, have been developed and used to treat hyperlipidemia. Fibrates are a class of amphipathic carboxylic acid compounds which are agonists of peroxisome proliferator-activated receptor-alpha (PPARα) and structurally contain aromatic groups as the lipophilic moiety and carboxylic acid or its ester as the hydrophilic moiety. Fibrates are used for a range of metabolic disorders, mainly hypercholesterolemia. Representative fibrates include, but are not limited to Bezafibrate (i.e., Bezalip®), Ciprofibrate (i.e., Modalim®), Clofibrate (i.e., Atromid-S), Gemfibrozil (i.e., Lopid®), and Fenofibrate (i.e., TriCor®). Statins are HMG-CoA reductase inhibitors which structurally contain multiple aromatic or other unsaturated rings, one or more hydroxyl groups, and carboxylic acid, ester, or lactone. Statins are widely used as pharmaceutical agents to lower cholesterol levels in people with or at risk for cardiovascular disease. Representative fibrates include, but are not limited to atorvastatin (i.e., Lipitor®), cerivastatin (i.e., Lipobay® or Baycol®), fluvastatin (i.e., Lescol® or Lescol XL®), lovastatin (i.e., Mevacor® or Altocor®), mevastatin, pitavastatin (i.e., Livalo® or Pitava®), pravastatin (i.e., Pravachol®, Selektine®, or Lipostat®), rosuvastatin (i.e., Crestor®), and simvastatin (i.e., Zocor®). LDL-lowering potency of statins varies between agents. Cerivastatin is the most potent, followed by (in order of decreasing potency) rosuvastatin, atorvastatin, simvastatin, lovastatin, pravastatin, and fluvastatin. However, cerivastatin was withdrawn from the market in 2001 due to the high rate of serious side-effects. The relative potency of pitavastatin has not yet been fully established.

Fibrates and statins are both hypolipidemic agents, but function through different biological mechanisms. Fibrates activate PPAR, especially PPARα, which is a class of intracellular receptors that modulate carbohydrate, fat metabolism and adipose tissue differentiation. Activation of PPARs causes transcription of a number of genes on the DNA that facilitate lipid metabolism. This increases synthesis of lipoprotein lipase therefore increasing clearance of triglycerides. Statins lower cholesterol by inhibiting the enzyme HMG-CoA reductase, which is the rate-limiting enzyme of the mevalonate pathway of cholesterol synthesis. Inhibition of this enzyme in the liver stimulates LDL receptors, resulting in an increased clearance of LDL from the bloodstream and a decrease in blood cholesterol levels. The first results can be seen after one week of use of a statin and the effect is maximal after four to six weeks. As shown in the table below, fibrates alter HDL and triglycerides levels more efficiently, while statins work better in regulating LDL—hence a combination of these drugs offers significant therapeutic benefits.

TABLE 1
Comparison of the Efficacies between Statins and Fibrates

| | LDL-Cholesterol | HDL-Cholesterol | |
|---|---|---|---|
| | (<<bad | (<<good | Triglycerides |
| | cholesterol>>) | cholesterol>>) | (TG) |
| Fibrates | Decreased by | Increased by | Decreased by |
| | 10-30% | 10-15% | 40-50% |
| Statins | Decreased by | Increased by | Decreased by |
| | 25-60% | 5-12% | 15-30% |

| | |
|---|---|
| Historically first | Increasingly important as independent |
| cardiovascular risk | risk factors for coronary heart disease, |
| factor investigated | especially in type 2 diabetes |

Fibrates in general and fenofibrate in particular are known to be poorly water-soluble. Poor water-solubility of an active pharmaceutical ingredient (API), such as a fibrate, always poses significant problems with respect to bioavailability when administered through oral or other routes of administration. This is due to difficulties in making the API bioavailable in aqueous biological systems. In the case of formulations intended for oral administration, poorly water-soluble APIs, such as fibrates, are susceptible to inadequate drug absorption, or are absorbed under wildly variable rates and/or extent of drug absorption (i.e., variable uptake between fed and fasted states). Fibrates, particularly fenofibrate, show low oral bioavailability and significant fed/fasted variability in the native forms. Some poorly water-soluble APIs are never commercialized because they cannot be effectively solubilized in the biologic milieu, and therefore fail to exhibit acceptable in vivo therapeutic activity. Alternatively, the quantity of poorly water-soluble API required to be administered to achieve an acceptable level of therapeutic activity may be unreasonably high, given the poor water solubility of the agent, and possibly resulting in unacceptable toxicity.

Statins have a wide range of bioavailability and food interactions. See, for example, American Family Physician, 2002, Vol. 65, No. 6, page 1173. For example, simvastatin is less than 5 percent bioavailable, while fluvastatin is approximately 24 percent bioavailable. Furthermore, statins are absorbed at rates ranging from about 30 percent with lovastatin to 98 percent with fluvastatin. With respect to food interactions, the bioavailability of lovastatin increases under fed conditions, while the bioavailability of pravastatin, atorvastatin, and fluvastatin decrease under fed conditions. The variation of bioavailability and food interactions among statins certainly complicates the selection of statins for treating patients.

In a combination dosage form of a statin and a fibrate such as fenofibrate, intake or absence of intake of food can lead to unexpectedly high or low levels of the active fibrate in the presence of a given dosage level of a statin. This lack of control of fibrate level in the blood can potentially lead to undesired side effects such as myopathy and rhabdomyolysis that have sometimes been seen previously with statins alone and with fibrates and statins when administered concurrently to a patient, particularly as a result of concurrent administration of gemfibrozil and lovastatin. Administration of separate dosage forms of a statin and of a fibrate can also pose the potential for variable uptake of either drug, for example when a patient overdoses or underdoses one or the other individual dosage form by taking more or fewer doses of either separate drug than the patient's condition would require for treatment. This can happen when a patient forgets to take one or the other drug dosage form, or when the patient forgets that he or she has taken one or the other drug dosage form and subsequently takes a second or even a third or more dosage form of one or both of the drugs. This can be especially prevalent in an older patient and in a patient with a failing memory.

Reduction of the particle size of the API can result in increased surface area, which may result in greater water solubility and/or better dissolution properties. Techniques such as microprecipitation, micronization, milling, homogenization, super critical fluid particle generation, etc. have been used to reduce particle sizes of APIs. Exemplary milling techniques typically include dry and wet milling. However, dry milling does not offer additional benefits such as surface stabilization, increased wettability, or improved dispersion properties, and wet milling can be cost prohibitive for a number of APIs. There are several commercial technologies to address these issues namely NanoCrystal® technology from élan Drug Technologies, NanoEdge® technology from Baxter BioPharma Solutions, Insoluble Drug Delivery (IDD®) Technology from Skye Pharma, and Biorise® technology from Eurand. However, even after micronization, there are high fed-fast variability of fibrates in their bioabsorption and bioavailability.

Thus, there is a strong need in the art for cost-effective methods of formulating a combined pharmaceutical composition of a fibrate and a statin into suitable dosage forms exhibiting optimal in vivo efficacy. Particularly, there is a need for oral dosage forms of a combined pharmaceutical composition of a fibrate and a statin which exhibit reduced fed/fasted absorption variability, or similar or bioequivalent absorption profiles when administered under fed and fasting conditions. It is also desirable that such oral dosage forms of a combined pharmaceutical composition of a fibrate and a statin can reduce the efficacious amount of the active pharmaceutical ingredients (“APIs”) used in the pharmaceutical compositions thereby reducing potential side effects of the APIs.

### SUMMARY OF THE INVENTION

In one aspect, the present invention provides a pharmaceutical formulation comprising a multi-phasic pharmaceutical composition in an oral dosage form. The multi-phasic pharmaceutical composition comprises: (a) a fibrate, or a pharmaceutically acceptable salt, ester, hydrate, active metabolite, or prodrug thereof; (b) a statin, or a pharmaceutically acceptable salt, ester, hydrate, active metabolite, or prodrug thereof; (c) a solvent; (d) a non-miscible liquid; (e) a stabilizer; and (f) water; wherein the fibrate or the statin or both are in a particulate state and/or a solubilized state.

In one embodiment of the present invention, the multi-phasic pharmaceutical composition comprises more than one stabilizers and the non-miscible liquid is non-water-miscible.

In one embodiment of the present invention, the pharmaceutical formulation further comprises an adsorbent carrier.

In one embodiment of the present invention, the multi-phasic pharmaceutical composition further comprises globules of the non-miscible liquid and the globules have a diameter of less than about 10 μm.

In one embodiment of the present invention, the multi-phasic pharmaceutical composition exhibits a reduced variability in the quantity of drug absorbed (mean AUC), and/or the rate of drug absorption (mean Cₘₐₓ and/or mean Tₘₐₓ) following administration of the multi-phasic pharmaceutical composition to a mammal under fed conditions as compared to fasting conditions.

### BRIEF DESCRIPTION OF THE DRAWINGS

FIG. 1 is a particle size distribution of fenofibrate raw material, with a mean particle size of 57 μm.

FIG. 2 is a particle size distribution of fenofibrate following particle size reduction using the methods of U.S. Provisional Patent Application No. 60/779,420, applicable to the present invention, with a mean fenofibrate nano-emulsion droplet size of 60 nm, and with 100% of the fenofibrate particles having a size of less than 3 μm. The content of U.S. Provisional Patent Application No. 60/779,420 is herein incorporated by references in its entirety.

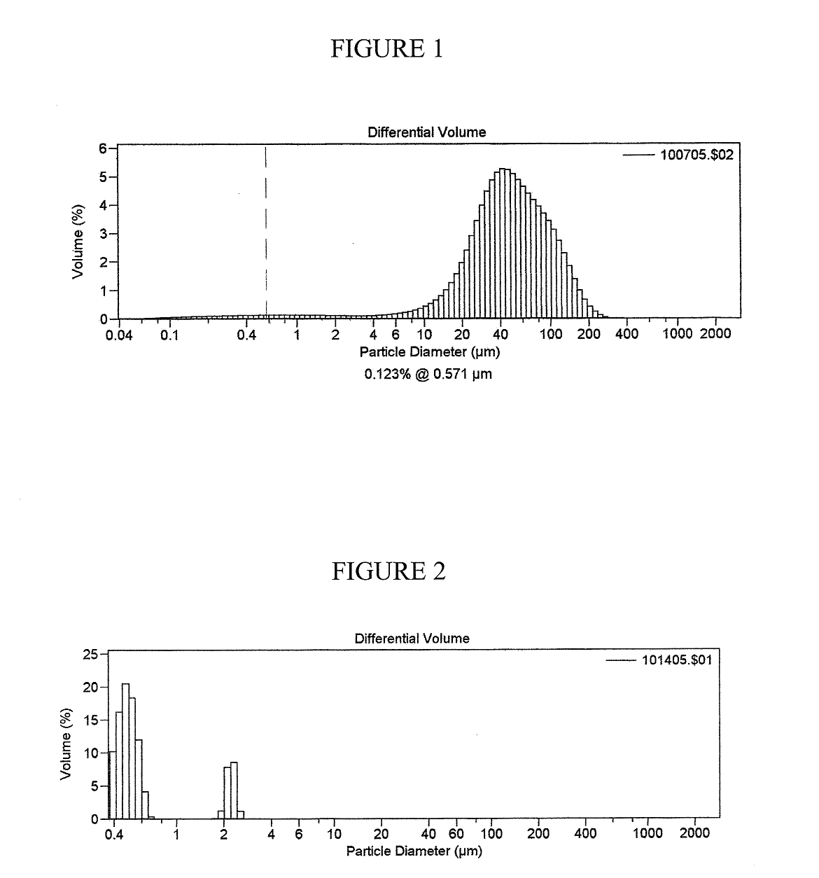

### DETAILED DESCRIPTION OF THE INVENTION

### A. Definitions

Unless stated otherwise, the following terms and phrases as used herein are intended to have the following meanings:

When trade names are used herein, applicants intend to independently include the tradename product and the active pharmaceutical ingredient(s) of the tradename product.

For the purposes of this disclosure and unless otherwise specified, the term “a” or “an” means “one or more.”

As used herein, the term “about” denotes being approximate or close a particular term, number, or numerical range. It is understood by persons of ordinary skill in the art that “about” may vary to some extent depending upon the context in which it is used. For example, given the context in which it is used, the term “about” may mean up to plus or minus 2%, 5%, or 10% of the particular term, number, or numerical range.

When a metabolite of a drug produces a therapeutic effect it is considered an “active metabolite”. A “metabolite” is any substance produced or used during metabolism or in a metabolic process, such as digestion. In drug use, the term “metabolite” usually refers to the substance(s) produced during metabolism of the drug.

As used herein, the term “adsorbent carrier” refers to materials, usually solid, employed to adsorb and/or absorb a liquid formulation.

The term “API” is an abbreviation for active pharmaceutical ingredient. As used herein, API includes both a compound and a pharmaceutically acceptable salt, ester, hydrate, active metabolite, or prodrug thereof.

The term “AUC” is an abbreviation for area under the curve. AUC is commonly used in pharmacokinetics to describe the quantity of drug absorbed by the recipient.

As used herein, the term “cellulose” includes the various forms of cellulose known for use in pharmaceutical formulations, including but not limited to, ethyl cellulose, cellulose acetate, carboxymethyl cellulose calcium, sodium carboxymethyl cellulose, methylcellulose, hydroxyethyl cellulose, hydroxypropyl cellulose, hydroxypropyl methylcellulose, Hydroxypropyl methylcellulose phthalate, microcrystalline cellulose, and mixtures thereof.

The term “Cₘₐₓ” refers to the maximum serum or blood concentration of a drug observed after its administration. Cₘₐₓ is commonly used in pharmacokinetics to describe the absorption profile, such as quantity or rate, of a drug in the recipient.

“Croscarmellose sodium” is cross-linked sodium carboxymethyl cellulose.

“Crospovidone” is a water-insoluble cross-linked homopolymer of 1-vinyl-2-pyrrolidinone.

By “Cyclodextrin”, it is meant a family of cyclic oligosaccharides containing at least six D-(+)-glucopyranose units.

As used herein, the term “emulsifier” refers to a material that promotes the formation of an emulsion and the term “emulsion” denotes a colloid formed by non-miscible liquids. Specifically, an emulsion refers to a dispersion of one non-miscible liquid in another liquid. For example, the non-miscible liquids of the present invention can form an emulsion with the solvents of the present invention.

“Ester thereof” means any ester of a fenofibrate or a statin or both in which any of the —COOH functions of the molecule is replaced by a —COOR function, in which the R moiety of the ester is any carbon-containing group which forms a stable ester moiety, including but not limited to alkyl, alkenyl, alkynyl, cycloalkyl, cycloalkylalkyl, aryl, arylalkyl, heterocyclyl, heterocyclylalkyl and substituted derivatives thereof. The term “ester thereof” includes but is not limited to pharmaceutically acceptable esters thereof.

By “fatty acid,” it is meant any of the members of a large group of monobasic acids, especially those found in animal and vegetable fats and oils. In some embodiments the fatty acid is straight or branched chain alkyl or alkenyl group having 6 to 22 carbons, wherein the carboxylic acid is at one terminus of the carbon chain.

As used herein, the term “globule” denotes a small spherical drop of fluid or semifluid substance, i.e., the non-miscible liquid in the solvent or water.

By “glycerides,” it is meant esters formed between one or more acids and glycerol. In some embodiments, the acids are fatty acids. Medium-chain glycerides are glycerol esters of medium-chain fatty acids containing from 6 to 12 carbon atoms, or, in some embodiments, 6 to 10 carbon atoms. Medium chain fatty acids include: caproic acid (C₆); caprylic acid (C₈), capric acid (C₁₀), and lauric acid (C₁₂). Long chain glycerides are glycerol esters of long chain fatty acids containing from 12 to 22 carbon atoms, or in some embodiments, 12 to 18 carbon atoms.

As used herein, “hypolipidemic agent” refers to any compound that promotes the reduction of lipid concentrations in the serum.

By “lipid,” it is meant any of a group of organic compounds, including, but not limited to the fats, oils, waxes, sterols, and triglycerides, that are insoluble in water but soluble in non-polar organic solvents, and are oily to the touch.

The term “micelle”, as used herein, denotes a molecular aggregate that constitutes a colloidal particle. A typical micelle is an aggregate of surfactant molecules dispersed in a liquid colloid. A micelle in aqueous solution usually forms an aggregate with the hydrophilic “head” regions in contact with surrounding solvent, sequestering the hydrophobic tail regions in the micelle centre. This type of micelle is known as a normal phase micelle (oil-in-water micelle). Inverse micelles have the headgroups at the centre with the tails extending out (water-in-oil micelle). Micelles are approximately spherical in shape. Other phases, including shapes such as ellipsoids, cylinders, and bilayers are also possible.

As used herein, “microsponge” refers to a porous material capable of adsorbing or absorbing liquids.

As used herein, the term “non-miscible liquid” refers to a liquid that does not dissolve in another liquid, for example, water and/or the solvents of the present invention. Non-miscible liquids are capable of forming emulsions. In one embodiment of the present invention, non-miscible liquids are capable of forming emulsions with water and/or the solvents of the present invention.

The term “particulate state,” as used herein, denotes insoluble particles of a given material. One example of such a given material is an API. Preferably, these insoluble particles are nano- or micro-particles.

“Pharmaceutically acceptable salt” means a salt of a fenofibrate or a statin or both which is, within the scope of sound medical judgment, suitable for use in contact with the tissues of humans and lower animals without undue toxicity, irritation, allergic response, and the like, commensurate with a reasonable risk/benefit ratio, generally water or oil-soluble or dispersible, and effective for their intended use. Where applicable and compatible with the chemical properties of the fenofibrate or statin, the term includes pharmaceutically-acceptable acid addition salts and pharmaceutically-acceptable base addition salts. Lists of suitable salts are found in, e.g., S. M. Birge et al., J. Pharm. Sci., 1977, 66, pp. 1-19.

By “non-phospholipid,” it is meant not being phospholipid. The term “phospholipid” refers to phosphorous-containing lipids that are composed mainly of fatty acids, a phosphate group, and a simple organic molecule, i.e., glycerol. Phospholipids may also be referred to as phosphatides.

As used herein, “poorly water-soluble” or “water insoluble” refers to materials, such as an API, that have a solubility in water of less than about 20 mg/mL, less than about 10 mg/mL, less than about 1 mg/mL, less than about 0.1 mg/mL, less than about 0.01 mg/mL, or less than about 0.001 mg/mL at ambient temperature and pressure, and at about pH 7.

“Povidone”, as used herein, is a polymer of 1-vinyl-2-pyrroldinone, and having a wide range of average molecular weight. In some embodiments, the povidone has an average molecular weight of from about 2,500 g/mol to about 300,000 g/mol, or greater.

The term “prodrug” as used herein refers to any compound that when administered to a biological system generates the drug substance, i.e., active ingredient, as a result of spontaneous chemical reaction(s), enzyme catalyzed chemical reaction(s), photolysis, and/or metabolic chemical reaction(s). A prodrug is thus a covalently modified analog or latent form of a therapeutically-active compound.

“Relative exposure” is a percentage value based upon AUC measurements. The percentage is calculated by assigning an AUC value, a value of 100% and expressing the other AUC values as a percentage of the 100% AUC value.

“Salt thereof” means any acid and/or base addition salt of a fenofibrate or a statin or both according to the invention; preferably a pharmaceutically acceptable salt thereof.

As used herein, the term “solubilized state” denotes a solution phase of a given material or an emulsion phase of a given material or both, wherein the solution phase comprises complete solubilization of the given material in a solvent, a non-miscible liquid, or water; and the emulsion phase comprises partial solubilization of the given material within the emulsion in which the given material is stabilized by micro- or nano-micelles. One example of such a given material is an API.

“Solvate thereof” means a fibrate or a stain or both formed by solvation (the combination of solvent molecules with molecules or ions of the solute), or an aggregate that consists of a solute ion or molecule (the fibrate or the stain or both) with one or more solvent molecules.

The term “sorbitan,” as used herein, refers to dehydrated Sorbitol.

The term “starch” refers to a complex carbohydrate consisting of amylose and amylopectin. “Pregelatinized starch” is starch that has been chemically and/or mechanically processed to rupture all or part of the granules in the presence of water and subsequently dried. Some types of pregelatinized starch may be modified to render them compressible and flowable in character.

The term “subject,” as used herein, refers to any animal that can experience the beneficial effects of the formulations and methods embodied herein. Preferably, the animal is a mammal, and in particular a human, although it is not intended to be so limited. Examples of other suitable animals include, but are not limited to, rats, mice, monkeys, dogs, cats, cattle, horses, pigs, sheep, and the like.

By “substantially similar to”, it is meant to a great extent/degree being similar to that which is specified.

The term “sugar fatty acid,” as used herein, refers to a fatty acid with a sugar moiety attached.

The term “Tₘₐₓ” refers to the time to reach Cₘₐₓ. Tₘₐₓ is commonly used in pharmacokinetics to describe the rate of drug absorption in a recipient.

As used herein, the term “therapeutically effective amount” with respect to an API dosage shall mean that dosage that provides the specific pharmacological response for which the API is administered in a significant number of subjects in need of such treatment. It is emphasized that “therapeutically effective amount,” administered to a particular subject in a particular instance may not be effective for 100% of patients treated for a specific disease, and will not always be effective in treating the diseases described herein, even though such dosage is deemed a “therapeutically effective amount” by those skilled in the art.

It will be readily understood by persons of ordinary skill in the art, that some materials identified below as belonging to a category such as an adsorbent carrier, polymeric carriers, phospholipid carriers, pharmaceutically acceptable additives, or other carriers or additives may fall into one or more of those categories, although the material is listed in only one category. For example, magnesium aluminum silicate is both an adsorbent carrier and a synthetic or semi-synthetic polymeric carrier. As another example, cellulose may be an adsorbent carrier and a polymeric carrier. Other such materials belonging in more than one category, but listed in only one category, will be readily identified by one of skill in the art.

### B. Multi-Phasic Compositions in Solid and Liquid Forms

In one aspect, the present invention provides a pharmaceutical formulation comprising a multi-phasic pharmaceutical composition in an oral dosage form. The multi-phasic pharmaceutical composition comprises: (a) a fibrate; (b) a statin; (c) a solvent; (d) a non-miscible liquid; (e) a stabilizer; and (f) water. The fibrate or the statin or both are in the multi-phasic pharmaceutical composition is in a particulate state and/or a solubilized state. Examples of fibarates suitable for the present invention include, but are not limited to bezafibrate, ciprofibrate, clofibrate, gemfibrozil, fenofibrate, fenofibric acid, and a mixture of any two or more thereof. Examples of statins suitable for the present invention include, but are not limited to atorvastatin, cerivastatin, fluvastatin, lovastatin, mevastatin, pitavastatin, pravastatin, rosuvastatin, simvastatin, and a mixture of any two more thereof. The fibrate and/or statin include one or more compounds in their free acid or base forms or pharmaceutically acceptable salts, esters, hydrates, active metabolites, or prodrugs thereof

Solvents useful in the embodied pharmaceutical formulations include, but are not limited to, an alcohol; N-methyl pyrrolidinone; methoxypolyethylene glycol; polyethylene glycol; polyethylene oxide; ethoxy diglycol; triacetin; dimethylsulfoxide; propylene glycol; isopropyl myristate; mono-, di-, or tri-glycerides; diethylene glycol monoethyl ether; and a mixture of any two or more thereof. Exemplary alcohols include, but are not limited to aliphatic or aromatic alcohols, such as benzyl alcohol, ethyl alcohol, or a mixture of any two or more thereof. Exemplary polyethylene glycols have an average molecular weight of about 200 g/mol or greater, and the methoxypolyethylene glycol has an average molecular weight of about 1000 g/mol or greater. In other embodiments, the polyethylene glycol has an average molecular weight of about 1000 g/mol or greater. In other embodiments, the polyethylene glycol has an average molecular weight of from about 1000 g/mol to about 20,000 g/mol, and the methoxypolyethylene glycol has an average molecular weight of from about 1000 g/mol to about 20,000 g/mol.

Non-miscible liquids for use in the embodied pharmaceutical formulations include, but are not limited to, mono-, di- and/or tri-glycerides of low, medium or long-chain fatty acids, medium chain glycerides, long chain glycerides, ethyl esters of a fatty acid, propylene glycol fatty acid esters, sorbitan fatty acid esters, polyglyceryl fatty acid esters, glyceryl mono-, di-, or tri-caprylic acid esters; glyceryl mono-, di-, or tri-capric acid esters; or a mixture of any two or more thereof. Non-miscible liquids also include vegetable oils, nut oils, fish oils, lard oil, mineral oils, squalane, tricaprylin (1,2,3-trioctanoyl glycerol), and mixtures of any two or more thereof. For example, almond oil (sweet), apricot seed oil, borage oil, canola oil, coconut oil, corn oil, cotton seed oil, fish oil, jojoba bean oil, lard oil, linseed oil (boiled), macadamia nut oil, medium chain triglycerides, mineral oil, olive oil, origanum oil, peanut oil, safflower oil, sesame oil, soybean oil, sunflower seed oil, wheat germ oil, mineral oil (light), DL-α-tocopherol, ethyl oleate, ethyl linoleate, glyceryl behenate, glyceryl monooleate, glyceryl monostearate, glyceryl palmitostearate, linoleic acid, linolenic acid, oleic acid, palmitostearic acid, peppermint oil, polyglyceryl oleate, propylene glycol monolaureate, propylene glycol dilaureate, sorbitan monolaurate, sorbitan monooleate, sorbitan monopalmitate, sorbitan monostearate, sorbitan trioleate, stearic acid, tetraglyceryl monooleate, or a mixture of any two or more thereof are all examples of non-miscible liquids for use in the embodied pharmaceutical formulations. In preferred embodiments of the present invention, the non-miscible liquids are non-water-miscible, i.e., not miscible with water.

Stabilizers useful in the embodied pharmaceutical formulations include, but are not limited to, non-phospholipid surfactants, non-phenol polyethylene glycol ethers, sorbitan esters, polyethylene glycol esters, block polymers, acrylic polymers, ethoxylated fatty acids, ethoxylated alcohols, ethoxylated fatty acid esters, monoglycerides, silicon-based surfactants, polysorbates, tergitols, sugar fatty acid ester; a sucrose mono-, di-, or tri-fatty acid ester; a polyoxyethylene castor oil compound; a polyoxyethylene sorbitan fatty acid ester; a polyoxyethylene mono- or di-fatty acid ester; a polyoxyethylene alkyl ether; a glyceryl mono-, di-, or tri-fatty acid ester; a mixtures of polyoxyethylene mono- or di-ester of a C₈-C₂₂ fatty acid; a glyceryl mono-, di-, or tri-ester of a C₈-C₂₂ fatty acid, or a mixture of any two or more thereof. For example, the stabilizer may be ARLACEL™, BRIJ™, Cremophore RH-40, glycerin monostearate, PEMULEN™, Pluronics™, polyethylene glycol stearate, polyoxyl 35 castor oil, polyoxyl 40 hydrogenated castor oil, polyoxyl 60 hydrogenated castor oil, polysorbate 20, polysorbate 40, polysorbate 60, polysorbate 80, polyoxyl 40 stearate, polyoxyl 40 oleate, polyoxyl 20 cetostearyl ether, polyoxyl 10 oleyl ether, sodium dioctyl sulfosuccinate, sodium lauryl sulfate, SPAN™, TERGITOL™ NP-40, TERGITOL™ NP-70, DL-α-tocopheryl polyethylene glycol succinate, TWEEN™ 20, TWEEN™ 60, TWEEN™ 80, or a mixture of any two or more thereof. In embodiments of the present invention, the pharmaceutical formulations comprise one or more stabilizers.

Liquid form drug compositions are ubiquitous throughout the pharmaceutical industry, existing as compositions of solutions, suspensions, emulsions, and the like. While liquid dosage forms are convenient forms, especially for pediatric and geriatric applications, conversion of these liquid compositions to a solid dosage form (i.e., tablets or capsules) can add significantly to both patient compliance and the commercial value to the products. Simple aqueous-based solutions or suspensions may be converted to a corresponding solid dosage form by, for example, lyophilizing with suitable cryoprotectants, the resulting mass being mixed with one or more suitable diluents, followed by filling into capsules or compressing into tablets.

In instances where the oral dosage form is a solid dosage form, the pharmaceutical formulations embodied herein comprise a multi-phasic pharmaceutical composition and an adsorbent carrier. Without being bound by theory, adsorbent carriers adsorb the non-miscible liquid (in some embodiments, an oil) that is present in the multi-phasic pharmaceutical composition to aid in the formation of a solid dosage form pharmaceutical formulation. Suitable adsorbent carriers for use in the embodied pharmaceutical formulations include porous materials, clays, silicates, cellulose-based polymers, microsponges, other synthetic polymers, or mixtures of any two or more thereof. Exemplary clays include attapulgite, bentonite, kaolin, perlite, talc, vermiculites, zeolites, or a mixture of any two or more thereof. Exemplary silicates include aluminum silicate, magnesium aluminum silicate, hydrous calcium silicate, colloidal silicon dioxide, magnesium aluminometasilicate, and mixtures of any two or more thereof. Exemplary cellulose-based polymers include carboxymethyl cellulose calcium, carboxymethyl cellulose sodium, cellulose, cellulose acetate, cellulose acetate phthalate, ethyl cellulose, hydroxyethyl cellulose, hydroxypropyl cellulose, hydroxypropyl methylcellulose, hydroxypropyl methylcellulose phthalate, methylcellulose, microcrystalline cellulose, powdered cellulose, or a mixture of any two or more thereof. Other synthetic polymers suitable for use as adsorbent carriers include cross-linked acrylic polymers, polypropylene, polyurethane foams, or mixtures of any two or more thereof.

Other adsorbent carriers that may be used in the embodied solid dosage forms include, but are not limited to, calcium carbonate, calcium phosphate dibasic anhydrous, calcium phosphate dibasic dehydrate, calcium phosphate tribasic, calcium sulfate, lactose, magnesium carbonate, magnesium oxide, mannitol, silicon dioxide, sodium starch glycolate, sodium chloride, sorbitol, starch, sucrose, or a mixture of any two or more thereof.

Additional excipients, carriers, and additives may also be included in the embodied solid dosage forms. Such other carriers and additives may be used to give binding, coloring, compressing, filling, flavoring, lubricating, and/or preserving properties to the pharmaceutical formulations or they may be used for other purposes known to those of skill in the art. For example, other carriers and additives may include, but are not limited to polymeric carriers, phospholipid carriers, lubricants, antioxidants, coloring agents, flavoring agents, preservatives, sweeteners, volatile oils, and/or a mixture of any two or more thereof. As used herein, the terms “excipients”, “carriers”, and “additives” are inter-exchangeable.

Exemplary polymeric carriers that may be used in the embodied pharmaceutical formulations include, but are not limited to, carbomers, croscarmellose sodium, crospovidone, cyclodextrins, β-cyclodextrins, Docusate sodium, hydroxypropyl-β-cyclodextrins, γ-cyclodextrins, polyanionic-β-cyclodextrins, sulfobutylether-7-β-cyclodextrin, methacrylic acid copolymers, poloxamer, polydextrose, polyethylene oxide, polymethacrylate polymers, poly(methacrylic acid-methyl methacrylate), poly(methacrylic acid-ethyl acrylate), ammonio methacrylate copolymer, poly(ethyl acrylate-methylmethacrylate-trimethylammonioethyl methacrylate chloride), poly(ethyl acrylate-methyl methacrylate), polysaccharides, polyvinyl alcohol with an average molecular weight of from about 20,000 to about 200,000 g/mol, polyvinylpyrrolidine/vinyl acetate, povidone with an average molecular weight of from about 2,500 to about 300,000 g/mol, sodium starch glycolate, or a mixture of any two or more thereof. Exemplary polysaccharides include, but are not limited to, acacia, alginic acid, carrageenan, ceratonia, chitosan, compressible sugar, confectioner's sugar, dextrates, dextrin, dextrose, fructose, fumaric acid, gelatin, glucose, glyceryl behenate, guar gum, lactitol, lactose, maltodextrin, maltose, mannitol, polydextrose, polymethacrylates, pregelatinized starch, sodium alginate, sorbitol, starch, sterilizable maize, sucrose, sugar spheres, tragacanth, trehalose, xylitol, or a mixture of any two or more thereof.

Some of the polymeric carriers may also be variously known in the art as disintegrants, compression aids, or binders. For example, disintegrants may include, but are not limited to, cellulose-based polymers; polysaccharides; other materials such as croscarmellose sodium, crospovidone, docusate sodium, magnesium aluminum silicate, colloidal silicon dioxide, calcium phosphate tribasic, povidone; or a mixture of any two or more thereof, as well as other materials and mixtures known to those of skill in the art to be useful as disintegrants. Compression aids may include, but are not limited to, polysaccharides and cellulose-based polymers and also non-polymeric materials such as inorganic salts, including but not limited to, calcium carbonate, calcium phosphate, calcium sulfate, magnesium carbonate, magnesium oxide, sodium chloride. Binders may also include materials such as polysaccharides and other synthetic or semi-synthetic polymers.

Exemplary phospholipid carriers that may be used in the embodied pharmaceutical formulations include, but are not limited to, diphosphatidylglycerol, glycolipids, phosphatidic acid, phosphatidylcholine, phosphatidylethanolamine, phosphatidylglycerol, phosphatidylinositol, phosphatidylserine, sphingomyelin, or a mixture of any two or more thereof. Exemplary lubricants include magnesium stearate, talc, stearic acid, calcium stearate, zinc stearate, glyceryl palmitostearate, glyceryl behenate, light mineral oil, micronized poloxamers, polyethylene glycol, 1-leucine, vegetable oil.

The liquid and/or solid dosage forms embodied herein may also include pharmaceutically acceptable additives such as, but not limited to, an antioxidant, a coloring agent, a flavoring agent, a preservative, a sweetener, a volatile oil, or a mixture of any two or more thereof. Exemplary antioxidants include, but are not limited to, ascorbic acid, ascorbyl palmitate, butylated hydroxyanisole, butylated hydroxytoluene, ethylenediaminetetraacetic acid, salts of ethylenediaminetetraacetic acid, propyl gallate, sodium metabisulfite, vitamin E, esters of vitamin E, or a mixture of any two or more thereof. Exemplary preservatives include, but are not limited to, butylparaben, calcium sorbate, ethylparaben, methylparaben, monothioglycerol, potassium sorbate, propylparaben, sodium benzoate, sodium sorbate, sorbic acid, or a mixture of any two or more thereof. Exemplary sweeteners include, but are not limited to, aspartame, glycyrrhizin salts, monoammonium glycyrrhizinate, saccharin, saccharin calcium, saccharin sodium, sugar, sucralose, or a mixture of any two or more thereof. Exemplary flavoring agents include, but are not limited to, anise, banana, cherry, chocolate, citric acid, lemon, menthol, orange, peppermint, pineapple, rum, sodium citrate, strawberry, vanillin, ethyl vanillin, or a mixture of any two or more thereof. Exemplary coloring agents include, but are not limited to, FD&C blue #1, FD&C blue #2, FD&C green #3, FD&C red #3, FD&C red #4, FD&C yellow #5, FD&C yellow #6, D&C blue #4, D&C green #5, D&C green #6, D&C orange #4, D&C orange #5, iron oxides, or a mixture of any two or more thereof. Exemplary volatile oils include, but are not limited to, balm oil, bay oil, bergamot oil, cedarwood oil, cherry oil, cinnamon oil, clove oil, origanum oil, peppermint oil, or a mixture of any two or more thereof.

The oral dosage forms of the pharmaceutical formulations embodied herein, may be in solid or liquid dosage forms. Such solid or liquid forms may be formulated into suitable dosage forms known to those of skill in the art such as a capsule, emulsion, tablet, and the like. In some embodiments, the multi-phasic pharmaceutical composition is present in the pharmaceutical formulation at about 0.1 wt % to about 90 wt %. In some embodiments, the API, i.e., the combination of a fibrate and a statin, is present in the pharmaceutical formulation at about 0.1 to about 70 wt %. The conversion of liquid multi-phasic preparations into an oral solid dosage form has been described in the U.S. Provisional Application No. 60/857,511 (entitled “Method of Preparing Solid Dosage Forms of Multi-Phasic Pharmaceutical Compositions”), which is herein incorporated by reference in its entirety.

The use of solid dosage forms such as capsules, tablets, lozenges, and/or cachets is well known in the art for the oral, buccal, or rectal administration of a pharmaceutical agent to a subject. The pharmaceutical formulations embodied herein, may be used in the preparation of such capsules, tablets, lozenges, and/or cachets. Capsules may be hard or soft, and may be made of a variety of materials known to those of skill in the art, including, but not limited to, cellulose materials, gelatin, carrageenan, agar, and pectin. When such solid dosage forms are placed in aqueous media, the formulations disintegrate to release the active pharmaceutical ingredient.

The use of liquid dosage forms such as solutions, emulsions, suspensions, syrups, elixirs, capsules, and the like is well known in the art for the oral administration of a pharmaceutical agent to a subject. The pharmaceutical formulations embodied herein, may be used in the preparation of such solutions, emulsions, capsules, and the like. Capsules may be hard or soft, and may be made of a variety of materials known to those of skill in the art, including, but not limited to, cellulose materials, gelatin, carrageenan, agar, and pectin.

### C. Multi-Phasic Compositions and Fed/Fasted Variability

Many administered drugs, especially those in oral dosage forms, are susceptible to bioavailability variations due to the presence or absence of food in the subject's digestive system. Such variability may be evidenced by changes in the AUC, Tₘₐₓ, Cₘₐₓ, or relative exposure when comparing values determined for a subject before and after feeding. The multi-phasic compositions of the present invention may be used to significantly reduce, or in some cases eliminate, such variability for a wide range of drugs.

Multi-phasic compositions are versatile vehicles for a wide variety of active pharmaceutical ingredients, and can be used for, but not limited to, the delivery of poorly water-soluble compounds, such as fibrates. For example, poorly water-soluble pharmaceuticals tend to be very difficult to deliver to a patient. However, multi-phasic compositions comprising both particulate state API and solubilized state API provide a new route for oral, buccal, vaginal, intranasal, parenteral, or rectal administration for such pharmaceuticals.

Multi-phasic compositions may be described in some embodiments as comprising a drug that is distributed in different phases, or forms, within the same composition, for example as a micro- or nano-particulate form, and/or as a solubilized form (within i.e., an oil, solvent, and/or micelle). Such compositions present the API with significantly enhanced the surface area—principally due to its distribution within multiple phases (i.e., solid micro- or nano-particle, micro- or nano-emulsion and/or micro- or nano-micelle). In various embodiments, the API, such as a fibrate, a statin, or both, are: (i) completely soluble, (ii) completely insoluble, and/or (iii) partially soluble within the vehicle of the emulsion and/or micelle. This phase variation aids in improving bioavailability and in reducing fed/fast variability in oral dosage formulations. Furthermore, the amount of the APIs required for producing efficacious effects can be reduced due to the increased bioavailability and reduced fed/fast variability. The reduced dosage of the APIs can subsequently reduce any side effects of the APIs, such as, for example, rosuvastatin and cerivastatin. In some embodiments, the oral dosage formulation is a solid dosage form, and in other embodiments it is a liquid dosage form. Additional details of multi-phasic compositions are disclosed in the U.S. Provisional Application No. 60/881,470 (entitled: “Multi-Phasic Pharmaceutical Formulations of Poorly Water-Soluble Drugs for Reduced Fed/Fasted Variability and Improved Oral Bioavailability”), which is herein incorporated by reference in its entirety.

In some embodied methods and in the embodied pharmaceutical formulations, the multi-phasic composition comprises globules of the non-miscible liquid and the globules have a diameter of less than about 10 μm. For example, the globules may have a diameter of less than about 9 microns, less than about 8 microns, less than about 7 microns, less than about 6 microns, less than about 5 microns, less than about 4 microns, less than about 3 microns, less than about 2 microns, less than about 1000 nm, less than about 900 nm, less than about 800 nm, less than about 700 nm, less than about 600 nm, less than about 500 nm, less than about 400 nm, less than about 300 nm, less than about 290 nm, less than about 280 nm, less than about 270 nm, less than about 260 nm, less than about 250 nm, less than about 240 nm, less than about 230 nm, less than about 220 nm, less than about 210 nm, less than about 200 nm, less than about 190 nm, less than about 180 nm, less than about 170 nm, less than about 160 nm, less than about 150 nm, less than about 140 nm, less than about 130 nm, less than about 120 nm, less than about 110 nm, less than about 100 nm, less than about 90 nm, less than about 80 nm, less than about 70 nm, less than about 60 nm, less than about 50 nm, less than about 40 nm, less than about 30 nm, less than about 20 nm, or less than about 10 nm.

In some embodied methods and pharmaceutical formulations, the multi-phasic composition comprises at least a portion of the API, i.e., the fibrate or the statin or both, in particulate form. In some embodiments, the average diameter of the particles of the particulate form is from about 1 nm to about 10 microns. In some embodiments, the average diameter of the particles of the particulate form is less than about 10 microns. For example, the average diameter of the particles may be less than about 9 microns, less than about 8 microns, less than about 7 microns, less than about 6 microns, less than about 5 microns, less than about 4 microns, less than about 3 microns, less than about 2 microns, or about 1 micron or greater. In other embodiments, the average diameter of the particles is less than about 1 micron, such as from about 1 nm to about 1 micron. For example, the diameter of the API particles, i.e., the particles of the fibrate or the statin or both, may be less than about 900 nm, less than about 800 nm, less than about 700 nm, less than about 600 nm, less than about 500 nm, less than about 400 nm, less than about 300 nm, less than about 290 nm, less than about 280 nm, less than about 270 nm, less than about 260 nm, less than about 250 nm, less than about 240 nm, less than about 230 nm, less than about 220 nm, less than about 210 nm, less than about 200 nm, less than about 190 nm, less than about 180 nm, less than about 170 nm, less than about 160 nm, less than about 150 nm, less than about 140 nm, less than about 130 nm, less than about 120 nm, less than about 110 nm, less than about 100 nm, less than about 90 nm, less than about 80 nm, less than about 70 nm, less than about 60 nm, less than about 50 nm, less than about 40 nm, less than about 30 nm, less than about 20 nm, or less than about 10 nm.

In one embodiment of the invention, the difference between the AUC, Cₘₐₓ, Tₘₐₓ, or any combination thereof, of a drug administered under fasting conditions, as compared to the same drug (and same drug quantity) administered under fed conditions, preferably administered to a mammal such as a human, is less than about 1000%, less than about 900%, less than about 800%, less than about 700%, less than about 600%, less than about 500%, less than about 400%, less than about 300%, less than about 200%, less than about 100%, less than about 90%, less than about 80%, less than about 70%, less than about 60%, less than about 50%, less than about 40%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, or less than about 1%.

In yet another embodiment of the invention, a composition of the invention administered under fed conditions is bioequivalent to the same composition administered under fasting conditions, to a mammal, such as a human. In another embodiment of the invention, “bioequivalency” is defined pursuant to regulatory guidelines. Under the United Stated Food and Drug Administration (U.S. FDA) guidelines, two products or methods are bioequivalent if the 90% Confidence Intervals (CI) for AUC and Cₘₐₓ are between 0.80 to 1.25 (Tₘₐₓ measurements are not relevant to bioequivalence for regulatory purposes). The European Medicine's Agency (EMEA) has recently adopted the U.S. FDA guidelines, as previously EMEA guidelines to show bioequivalency required a 90% CI for AUC of between 0.80 to 1.25 and a 90% CI for Cₘₐₓ of between 0.70 to 1.43.

Without being bound to such limitations, the examples provided below illustrate the extent to which the bioavailability variations may be reduced between a fed and a fasted state in rat models. Thus in some embodiments, where the API is fenofibrate and atorvastatin, the formulation, when tested in a rat or rat model, may provide a change in the mean AUC between a fed and a fasted state of less than about 90,000 h*ng/ml, less than about 85,000 h*ng/ml, less than about 80,000 h*ng/ml, less than about 75,000 h*ng/ml, less than about 70,000 h*ng/ml, less than about 65,000 h*ng/ml, or less than about 60,000 h*ng/ml.

Relative exposure may also be used to express fed/fasted variability. Thus, in some embodiments, where the active agent is any API described herein, including but not limited to where the active agent is fenofibrate, the formulation may provide a change in the relative exposure between a fed and a fasted state of less than about 1000%, less than about 900%, less than about 800%, less than about 700%, less than about 600%, less than about 500%, less than about 400%, less than about 300%, less than about 200%, less than about 100%, less than about 90%, less than about 80%, less than about 70%, less than about 60%, less than about 50%, less than about 40%, less than about 35%, less than about 30%, less than about 25%, less than about 20%, less than about 15%, less than about 14%, less than about 13%, less than about 12%, less than about 11%, less than about 10%, less than about 9%, less than about 8%, less than about 7%, less than about 6%, less than about 5%, less than about 4%, or less than about 3%.

In other embodiments, the invention provides for formulations wherein: (a) the API is any active agent described herein, including but not limited to a fibrate, a statin, or both; and (b) when administered to a mammal, the formulation provides a minimal difference in the mean AUC, mean Cₘₐₓ, and/or mean Tₘₐₓ between a fed and a fasted state.

In yet other embodiments, when tested in a rat or a rat model, a formulation of the invention exhibits a difference in the mean AUC between a fed and a fasted state selected from the group consisting of less than about 90,000 h*ng/ml, less than about 85,000 h*ng/ml, less than about 80,000 h*ng/ml, less than about 75,000 h*ng/ml, less than about 70,000 h*ng/ml, less than about 65,000 h*ng/ml, less than about 60,000 h*ng/ml, less than about 55,000 h*ng/ml, less than about 50,000 h*ng/ml, less than about 45,000 h*ng/ml, less than about 40,000 h*ng/ml, less than about 35,000 h*ng/ml, less than about 30,000 h*ng/ml, less than about 25,000 h*ng/ml, less than about 20,000 h*ng/ml, less than about 15,000 h*ng/ml, and less than about 10,000 h*ng/ml.

The differences in AUC between fed and fasted states may be expressed in a number of ways, including, but not limited to a percentage difference between any two determined AUC values, or the difference between relative exposure values. Thus, in some embodiments, upon administration to a mammal, a percent difference between a mean AUC, mean Cₘₐₓ, and/or mean Tₘₐₓ determined at a fasted state and a mean AUC, mean Cₘₐₓ, and/or mean Tₘₐₓ determined at a fed state is less than about 1000%, less than about 900%, less than about 800%, less than about 700%, less than about 600%, less than about 500%, less than about 400%, less than about 300%, less than about 200%, less than about 100%, less than about 90%, less than about 80%, less than about 70%, less than about 60%, less than about 50%, less than about 40%, less than about 30%, less than about 20%, less than about 10%. In other embodiments, the percent difference is selected from the group consisting of less than about 9%, less than about 8%, less than about 7%, less than about 6%, less than about 5%, less than about 4%, less than about 3%, less than about 2%, less than about 1%, and less than about 0.05%.

In other embodiments of the invention, the invention provides for formulations wherein: (a) the API can be, but is not limited to, a fibrate, a statin, or both; and (b) upon administration to a mammal, the formulation exhibits a difference in the relative exposure between a fed and a fasted state of less than about 1000%. In yet other embodiments, the formulation exhibits a difference in the relative exposure between a fed and a fasted state selected from the group consisting of less than about 900%, less than about 800%, less than about 700%, less than about 600%, less than about 500%, less than about 400%, less than about 300%, less than about 200%, less than about 100%, less than about 90%, less than about 80%, less than about 70%, less than about 60%, less than about 50%, less than about 45%, less than about 40%, less than about 35%, less than about 30%, less than about 25%, less than about 20%, less than about 15%, less than about 10%, less than about 5%, less than about 4%, and less than about 3%.

One of ordinary skill will appreciate that effective amounts of a fibrate and/or a statin in the present pharmaceutical formulation can be determined empirically and can be employed in pure form or, where such forms exist, in pharmaceutically acceptable salt, hydrate, ester, active metabolite, or prodrug form. Actual dosage levels of the fibrate and/or the statin in the nanoparticulate compositions of the invention may be varied to obtain an amount of the API that is effective to obtain a desired therapeutic response for a particular composition and method of administration. The selected dosage level therefore depends upon the desired therapeutic effect, the route of administration, the potency of the administered API, the desired duration of treatment, and other factors.

Dosage unit compositions may comprise such amounts or submultiples thereof as may be used to make up the daily dose. It will be understood, however, that the specific dose level for any particular patient will depend upon a variety of factors: the type and degree of the cellular or physiological response to be achieved; activity of the specific agent or composition employed; the specific agents or composition employed; the age, body weight, general health, sex, and diet of the patient; the time of administration, route of administration, and rate of excretion of the agent; the duration of the treatment; drugs used in combination or coincidental with the specific agent; and like factors well known in the medical arts.

One skilled in the art will readily realize that all ranges and ratios discussed can and do necessarily also describe all subranges and subratios therein for all purposes and that all such subranges and subratios also form part and parcel of this invention. Any listed range or ratio can be easily recognized as sufficiently describing and enabling the same range or ratio being broken down into at least equal halves, thirds, quarters, fifths, tenths, etc. As a non-limiting example, each range or ratio discussed herein can be readily broken down into a lower third, middle third and upper third, etc.

All publications, patent applications, issued patents, and other documents, if any, referred to in this specification are herein incorporated by reference as if each individual publication, patent application, issued patent, or other document was specifically and individually indicated to be incorporated by reference in its entirety, for all purposes. Definitions that are contained in text incorporated by reference are excluded to the extent that they contradict definitions in this disclosure.

The present invention, thus generally described, will be understood more readily by reference to the following examples, which are provided by way of illustration and are not intended to be limiting of the present invention.

### EXAMPLES

### Formulation I: Control

Non-micronized fenofibrate powder was suspended in hydroxypropyl methylcellulose (HPMC, grade E4M) to give a 0.5 wt % suspension (48 mg of fenofibrate per gram of suspension). The suspension was mixed very well to ensure a uniform suspension free from lumps and/or aggregates.

### Formulation II: Standard

A TriCor tablet (48 mg fenofibrate per tablet, available from Abbott Pharma) was powered using a mortar and pestle to until an aggregate-free mass was obtained. The mass was then suspended in one milliliter of purified water to obtain a uniform suspension.

### Formulation III: Test I

Fenofibrate (4.8 gm) was mixed with ethanol (8.8 gm), polysorbate 80 (9.4 gm), and soybean oil (50.2 gm). Water (26.8 gm) was added and the entire mixture subjected to emulsification using a paddle-type stirrer. The resultant emulsion was then subjected to high-pressure homogenization (APV-1000) at 10,000 psi for three cycles.

### Formulation IV: Test II

Fenofibrate (4.8 g) was mixed with ethanol (15 g) and medium chain triglycerides (40.0 g, Crodamol GTCC). The mixture was warmed (40° C.) to dissolve the fenofibrate with gentle mixing. Separately, poloxamer 188 (7.0 g) was dissolved in water (33.2 g) to form a solution which was then added to the fenofibrate solution. The resulting mixture was subjected to emulsification using a paddle-type stirrer. The emulsion was further subjected to high-pressure homogenization (APV-1000) at 10,000 psi for three cycles.

### Preclinical Study

The investigation was carried out in rats by administering Formulations I-IV using an oral gavage at a dose of 90 mg fenofibrate per kilogram body weight of animal, and then observing the blood concentrations of fenofibric acid as a function of time. Fenofibric acid is the active, primary metabolite resulting from the administration of fenofibrate to a subject.

### Phase I. Demonstration of Improved Bioavailability Compared to Control

In the first phase, the control formulation, the standard formulation, and the Test I formulations were compared under fasted conditions. Five rats per group were used in this initial study. Each rat was given a single dose of 90 mg/kg of fenofibrate. The area under the plasma concentration-time curve (AUC) over a 24-hour period (correlating to the amount of drug absorbed or bioavailability), Cₘₐₓ (maximum concentration of the drug in the blood), and Tₘₐₓ (time to reach Cₘₐₓ) were measured for each of the three groups, and the data is presented in Table 2 below.

TABLE 2
Fenofibrate Bioavailability Data in Fasted Rats

| | AUC_{24 h} | | Cₘₐₓ | | Tₘₐₓ | |
|---|---|---|---|---|---|---|
| Group | (hr*ng/mL) | | (ng/mL) | | (hr) | |
| (Formulation) | Mean | SD | Mean | SD | Mean | SD |
| Control | 216,542.1 | 125,241.2 | 31,080.0 | 7851.9 | 4.4 | 2.2 |
| Standard | 1,480,971.8^{1,2} | 333,521.8 | 180,600.0 | 34,121.8 | 3.6 | 2.6 |
| Test I | 912,679.9¹ | 161,665.7 | 132,500.0 | 19,710.4 | 4.0 | 0.0 |

¹Statistically significant increase compared to Control (p < 0.05, two-tailed t-test)
²Statistically significant increase compared to Test I (p < 0.05, two-tailed t-test)

The dose and mean AUC were then used to compute the relative exposure (%) for each group. “Relative exposure” represents the extent of overall bioavailability of an API in a subject. The relative exposure projects how test and control formulations perform with respect to the formulation which gave best results. In this case the formulation with the best results was the Standard formulation (100%) to which the other formulations are normalized. This data is presented in Table 3.

TABLE 3
Fenofibrate Relative Exposure in Fasted Rats

| Group | Dose | Mean AUC | |
|---|---|---|---|
| (Formulation) | (mg/kg) | (h*ng/mL) | Relative Exposure % |
| Standard | 90 | 1480971.8 | 100.0 |
| Test I | 90 | 912679.9 | 61.6 |
| Control | 90 | 216542.1 | 14.6 |

Tables 2 and 3 show that both the Standard and Test I formulations present an improvement in oral bioavailability of Fenofibrate when compared to the Control formulation. The Standard formulation offered statistically significant higher bioavailability as compared to the Test I formulation.

### Phase II: Elimination of Fed/Fast Variability

In the second phase, the Standard and the Test II Formulations were compared under fed and fasted conditions. Rats were divided into four groups of five rats each: (i) Standard—fasted, (ii) Standard—fed, (iii) Test II—fasted, and (iv) Test II—fed. Each of the formulations, in an amount equivalent to a fenofibrate dose of 90 mg/Kg, was administered as a single oral dose and the resulting blood pharmacokinetics were evaluated (Table 4).

TABLE 4
Fenofibrate Bioavailability in Fed and Fasted Rats

| | AUC_{24 h} | | Cₘₐₓ | | Tₘₐₓ | |
|---|---|---|---|---|---|---|
| | (h*ng/mL) | | (ng/mL) | | (hr) | |
| Group | Mean | SD | Mean | SD | Mean | SD |
| Standard - Fasted | 1245585 | 487453 | 146000 | 49432 | 3.6 | 0.9 |
| Standard - Fed | 1345108 | 311789 | 137200 | 26186 | 3.6 | 0.9 |
| Test II - Fasted | 1862671 | 480725 | 207000 | 28080 | 3.2 | 1.1 |
| Test II - Fed | 1919344 | 274560 | 189400 | 31501 | 3.2 | 1.1 |

As Table 4 illustrates, under both fed and fasted conditions, the difference between the AUC for each of the two formulations was statistically insignificant. In other words, for the Standard formulation there was no variation in bioavailability of fenofibrate in rats between the fed and fasted states. The same was true for the Test II formulation. As above, this data may be presented in terms of relative exposure, as shown in Table 5.

TABLE 5
Fenofibrate Relative Exposure in Fed and Fasted Rats

| | | | Relative Exposure |
|---|---|---|---|
| | Dose | Mean AUC | % All Groups |
| Group | (mg/kg) | (h*ng/mL) | Compared |
| Standard - Fasted | 90 | 1245585 | 64.9 |
| Standard - Fed | 90 | 1345108 | 70.1 |
| Test II - Fasted | 90 | 1862671 | 97.0 |
| Test II - Fed | 90 | 1919344 | 100.0 |

Table 5 clearly illustrates that Test II formulations have a higher relative exposure as compared to the Standard formulations. Expressed as AUC, the higher exposure from the Test II formulation offers a statistically significant improvement as compared to the Standard formulation. Therefore, it may be concluded that the Test II formulation has significant improvements over the currently marketed preparations for Fenofibrate in terms of improving oral bioavailability and reducing fed/fast variability.

### Preparation of Fenofibrate+Atorvastatin Formulation

A multi-phasic composition of fenofibrate and atorvastatin was obtained by using the procedures and ingredients described as follows: fenofibrate and atorvastatin were dissolved/dispersed in a combination of ethanol and triglycerides at about 40° C. followed by cooling to room temperature. Poloxamer 188 was dissolved in water at room temperature and the resulting solution was mixed with the solution of fenofibrate and atorvastatin through a paddle-type stirrer for 15 min. The resulting coarse emulsion was fed into a homogenizer (APV 1000) at 10,000 psi for three passes. This process afforded the multi-phasic composition of fenofibrate and atorvastatin in a liquid form. The conversion of this liquid multi-phasic preparations into an oral solid dosage form can be conducted by following the procedures described in the U.S. Provisional Application No. 60/857,511 (entitled “Method of Preparing Solid Dosage Forms of Multi-Phasic Pharmaceutical Compositions”), which is herein incorporated by reference in its entirety.

TABLE 6
Ingredients for the Formulation of Fenofibrate + Atorvastatin

| Ingredient | % (w/w) |
|---|---|
| Fenofibrate | 13.0 |
| Atorvastatin calcium | 2.0 |
| Ethanol | 15.0 |
| Poloxamer 188 | 5.0 |
| Medium chain triglycerides | 40.0 |
| Water | 25.0 |

### Preparation of Fenofibrate+Rosuvastatin Formulation

A multi-phasic composition of fenofibrate and rosuvastatin was obtained by using the ingredients described below and the procedures described above in Preparation of Fenofibrate+Atorvastatin Formulation except that Atorvastatin calcium was replaced with Rosuvastatin calcium.

TABLE 7
Ingredients for the Formulation of Fenofibrate + Rosuvastatin

| Ingredient | % (w/w) |
|---|---|
| Fenofibrate | 13.0 |
| Rosuvastatin calcium | 2.0 |
| Ethanol | 15.0 |
| Poloxamer 188 | 5.0 |
| Medium chain triglycerides | 40.0 |
| Water | 25.0 |

While the present invention has been particularly shown and described with respect to preferred embodiments thereof, it will be understood by those skilled in the art that the foregoing and other changes in forms and details may be made without departing from the spirit and scope of the invention. It is therefore intended that the present invention not be limited to the exact forms and details described and illustrated but fall within the scope of the appended claims.

We claim:

1. A pharmaceutical formulation comprising a multi-phasic pharmaceutical composition in an oral dosage form, wherein the multi-phasic pharmaceutical composition comprises:
(a) a fibrate, or a pharmaceutically acceptable salt, ester, hydrate, active metabolite, or prodrug thereof;
(b) a statin, or a pharmaceutically acceptable salt, ester, hydrate, active metabolite, or prodrug thereof;
(c) a solvent;
(d) a non-miscible liquid;
(e) a stabilizer; and
(f) water;
wherein the fibrate or the statin or both are in a particulate state and/or a solubilized state.

2. The pharmaceutical formulation of claim 1, wherein the multi-phasic pharmaceutical composition comprises more than one stabilizers, and the non-miscible liquid is non-water-miscible.

3. The pharmaceutical formulation of claim 1, wherein the fibrate is selected from the group consisting of bezafibrate, ciprofibrate, clofibrate, gemfibrozil, fenofibrate, fenofibric acid, and a mixture of any two or more thereof.

4. The pharmaceutical formulation of claim 1, wherein the statin is selected from the group consisting of atorvastatin, cerivastatin, fluvastatin, lovastatin, mevastatin, pitavastatin, pravastatin, rosuvastatin, simvastatin, and a mixture of any two more more thereof.

5. The pharmaceutical formulation of claim 1, wherein the solvent is selected from the group consisting of an alcohol, N-methyl pyrrolidinone, methoxypolyethylene glycol, polyethylene glycol, polyethylene oxide, ethoxy diglycol, triacetin, dimethylsulfoxide, propylene glycol, isopropyl myristate, mono-, di- or tri-glycerides, diethylene glycol monoethyl ether, or a mixture of any two or more thereof.

6. The pharmaceutical formulation of claim 5, wherein the alcohol is benzyl alcohol, ethyl alcohol, or a mixture of any two or more thereof.

7. The pharmaceutical formulation of claim 5, wherein the polyethylene glycol has an average molecular weight of about 200 g/mol or greater, and the methoxypolyethylene glycol has an average molecular weight of about 1000 g/mol or greater.

8. The pharmaceutical formulation of claim 1, wherein the non-miscible liquid is selected from the group consisting of a fatty acid, a medium chain glyceride, a long chain glyceride, an ethyl ester of a fatty acid, a propylene glycol fatty acid ester, a sorbitan fatty acid ester, a polyglyceryl fatty acid ester, a glyceryl mono-, di-, or tri-caprylic acid ester; a glyceryl mono-, di-, or tri-capric acid esters; mono-, di- and/or triglycerides of low, medium or long-chain fatty acids; or a mixture of any two or more thereof.

9. The pharmaceutical formulation of claim 1, wherein the non-miscible liquid is selected from the group consisting of vegetable oils, nut oils, fish oils, lard oil, mineral oils, squalane, tricaprylin (1,2,3-trioctanoyl glycerol), and a mixture of any two or more thereof.

10. The pharmaceutical formulation of claim 9, wherein the non-miscible liquid is almond oil (sweet), apricot seed oil, borage oil, canola oil, coconut oil, corn oil, cotton seed oil, fish oil, jojoba bean oil, lard oil, linseed oil (boiled), macadamia nut oil, medium chain triglycerides, mineral oil, olive oil, origanum oil, peanut oil, safflower oil, sesame oil, soybean oil, sunflower seed oil, wheat germ oil, mineral oil (light), DL-α-tocopherol, ethyl oleate, ethyl linoleate, glyceryl behenate, glyceryl monooleate, glyceryl monostearate, glyceryl palmitostearate, linoleic acid, linolenic acid, oleic acid, palmitostearic acid, peppermint oil, polyglyceryl oleate, propylene glycol monolaureate, propylene glycol dilaureate, sorbitan monolaurate, sorbitan monooleate, sorbitan monopalmitate, sorbitan monostearate, sorbitan trioleate, stearic acid, tetraglyceryl monooleate, or a mixture of any two or more thereof.

11. The pharmaceutical formulation of claim 1, wherein the stabilizer is selected from the group consisting of non-phospholipid surfactants, non-phenol polyethylene glycol ethers, sorbitan esters, polyethylene glycol esters, block polymers, acrylic polymers, ethoxylated fatty acids, ethoxylated alcohols, ethoxylated fatty acid esters, monoglycerides, silicon-based surfactants, polysorbates, tergitols, sugar fatty acid ester; a sucrose mono-, di-, or tri-fatty acid ester; a polyoxyethylene castor oil compound; a polyoxyethylene sorbitan fatty acid ester; a polyoxyethylene mono- or di-fatty acid ester; a polyoxyethylene alkyl ether; a glyceryl mono-, di-, or tri-fatty acid ester; a mixtures of polyoxyethylene mono- or di-ester of a C₈-C₂₂ fatty acid; a glyceryl mono-, di-, or tri-ester of a C₈-C₂₂ fatty acid, or a mixture of any two or more thereof.

12. The pharmaceutical formulation of claim 11, wherein the stabilizer is selected from the group consisting of ARLACEL™, BRIJ™, Cremophore RH-40, glycerin monostearate, PEMULEN™, PLURONIC™, polyethylene glycol stearate, polyoxyl 35 castor oil, polyoxyl 40 hydrogenated castor oil, polyoxyl 60 hydrogenated castor oil, polysorbate 20, polysorbate 40, polysorbate 60, polysorbate 80, polyoxyl 40 stearate, polyoxyl 40 oleate, polyoxyl 20 cetostearyl ether, polyoxyl 10 oleyl ether, sodium dioctyl sulfosuccinate, sodium lauryl sulfate, SPAN™, TERGITOL™ NP-40, TERGITOL™ NP-70, DL-α-tocopheryl polyethylene glycol succinate, TWEEN™20, TWEEN™ 60, TWEEN™ 80, or a mixture of any two or more thereof.

13. The pharmaceutical formulation of claim 1, further comprising an adsorbent carrier.

14. The pharmaceutical formulation of claim 13, wherein the adsorbent carrier is a clay, a silicate, a cellulose-based polymer, a microsponge, porous materials, other synthetic polymers, or a mixture of any two or more thereof.

15. The pharmaceutical formulation of claim 14, wherein the absorbent carrier is selected from the group consisting of attapulgite, bentonite, kaolin, perlite, talc, vermiculites, zeolites, aluminum silicate, magnesium aluminum silicate, hydrous calcium silicate, colloidal silicon dioxide, magnesium aluminometasilicate, carboxymethyl cellulose calcium, carboxymethyl cellulose sodium, cellulose, cellulose acetate, cellulose acetate phthalate, ethyl cellulose, hydroxyethyl cellulose, hydroxypropyl cellulose, hydroxypropyl methylcellulose, hydroxypropyl methylcellulose phthalate, methylcellulose, microcrystalline cellulose, powdered cellulose, a cross-linked acrylic polymer, a polypropylene, a polyurethane foam, calcium carbonate, calcium phosphate dibasic anhydrous, calcium phosphate dibasic dehydrate, calcium phosphate tribasic, calcium sulfate, lactose, magnesium carbonate, magnesium oxide, mannitol, silicon dioxide, sodium starch glycolate, sodium chloride, sorbitol, starch, sucrose, and a mixture of any two or more thereof.

16. The pharmaceutical formulation of claim 13, further comprising one or more excipients selected from the group consisting of polymeric carriers, phospholipid carriers, an antioxidant, a lubricant, a disintegrant, a coloring agent, a flavoring agent, a preservative, a sweetener, a volatile oil, or a mixture of any two or more thereof.

17. The pharmaceutical formulation of claim 16, wherein the multi-phasic pharmaceutical composition is present at about 0.1 to about 90 wt %.

18. The pharmaceutical formulation of claim 1, wherein the oral dosage form is a solid or liquid oral dosage form.

19. The pharmaceutical formulation of claim 18, wherein the solid dosage form is a capsule a tablet, a lozenge, or a cachet.

20. The pharmaceutical formulation of claim 18, wherein the liquid dosage form is a solution, an emulsion, a suspension, a syrup, an elixir, or a capsule.

21. The pharmaceutical formulation of claim 1, wherein the multi-phasic pharmaceutical composition further comprises globules of the non-miscible liquid and the globules have a diameter of less than about 10 μm.

22. The pharmaceutical formulation of claim 21, wherein the globules have a diameter of less than about 9 microns, less than about 8 microns, less than about 7 microns, less than about 6 microns, less than about 5 microns, less than about 4 microns, less than about 3 microns, less than about 2 microns, less than about 1000 nm, less than about 900 nm, less than about 800 nm, less than about 700 nm, less than about 600 nm, less than about 500 nm, less than about 400 nm, less than about 300 nm, less than about 290 nm, less than about 280 nm, less than about 270 nm, less than about 260 nm, less than about 250 nm, less than about 240 nm, less than about 230 nm, less than about 220 nm, less than about 210 nm, less than about 200 nm, less than about 190 nm, less than about 180 nm, less than about 170 nm, less than about 160 nm, less than about 150 nm, less than about 140 nm, less than about 130 nm, less than about 120 nm, less than about 110 nm, less than about 100 nm, less than about 90 nm, less than about 80 nm, less than about 70 nm, less than about 60 nm, less than about 50 nm, less than about 40 nm, less than about 30 nm, less than about 20 nm, or less than about 10 nm.

23. The pharmaceutical formulation of claim 1, wherein an average diameter of the particles of the particulate state is less than about 1 micron.

24. The pharmaceutical formulation of claim 1, wherein the multi-phasic pharmaceutical composition exhibits a reduced variability in the mean AUC, mean Cₘₐₓ, and/or mean Tₘₐₓ following administration of the multi-phasic pharmaceutical composition to a mammal under fed conditions as compared to fasting conditions.

25. The pharmaceutical formulation of claim 24, wherein upon administration to a mammal, the pharmaceutical formulation exhibits an absorption profile under fed conditions which is substantially similar to, or bioequivalent to, the absorption profile of the same pharmaceutical formulation administered under fasting conditions.
